(19) United States
(12) Patent Application Publication
(10) Pub. No.: US 2025/0072722 A1
(43) Pub. Date: Mar. 6, 2025

(54) MOLD FOR FABRICATING INTEGRATED ENDOSCOPIC PROBE, INTEGRATED ENDOSCOPIC PROBE, AND METHOD FOR FABRICATING THE SAME

(71) Applicant: Medimaging Integrated Solution, Inc., Hsinchu (TW)

(72) Inventors: SHANGYI WU, Hsinchu (TW); JIA-DE ZHOU, Hsinchu (TW)

(21) Appl. No.: 18/823,420

(22) Filed: Sep. 3, 2024

(30) Foreign Application Priority Data
Sep. 5, 2023 (TW) 112133745

(51) Int. Cl. A61B 1/00; B29C 33/00; B29K 101/10; B29L 31/00

(52) U.S. Cl. CPC A61B 1/0011; A61B 1/00165; B29C 33/0022; B29K 2101/10; B29L 2031/753

(57) ABSTRACT

A mold for fabricating an integrated endoscopic probe, and an integrated endoscopic probe, and a method for fabricating the same are provided. The mold is used to minimize the endoscopic distal tip, increase the output, and improve fabrication quality. The integrated endoscopic probe and the fabrication method thereof can reduce the size of the endoscopic distal tip, increase the efficiency and stability of the fabrication process, and reduce production cost.

### BACKGROUND OF THE INVENTION

### 1. Field of the Invention

The present invention provides an endoscopic probe and a fabrication method thereof, particularly a mold for fabricating an integrated endoscopic probe, an integrated endoscopic probe, and a fabrication method thereof.

### 2. Description of the Prior Art

At present, there are mainly two methods for fabricating an endoscopic distal tip. The first method includes steps: fabricating a U-shape endoscopic front end; placing an image sensing module, a light source module, a transmission cable, and a working channel into the endoscopic front end, which is pre-formed beforehand; filling a resin material into the endoscopic front end and curing the resin material to fix the abovementioned elements and then obtain an endoscopic distal tip structure. The pre-forming process is unlikely to perform unless the endoscopic front end has a specified thickness. Therefore, the pre-formed endoscopic front end must have a size over a threshold value. Thus, the pre-formed endoscopic front end, which is fabricated in the first method, has an intrinsic limitation in miniaturization. Hence, the first method for fabricating an endoscopic distal tip is unlikely to be applied to fabricating the endoscope having higher precision and smaller size.

The second method for fabricating an endoscopic distal tip includes steps: preparing a U-shape mold only having a single opening; sequentially placing elements, such as an image sensing module, a light source module, a transmission cable, and a working channel, into the U-shape mold; filling a resin material into the U-shape mold; and stripping off the U-shape mold to form an endoscopic distal tip structure. In the second method, the U-shape mold only has a single opening. While the image sensing module, the light source module, the transmission cable, and the working channel are to be sequentially placed into a limited space of the U-shape mold, the process must be elaborately controlled and thus consumes much time. Besides, the layout and configuration of the elements in the internal space would consume furthermore time. In the second method, the transmission cable, the image sensor and the illumination element must be electrically connected before they are placed into the U-shape mold. However, these electronic elements are hard to endure high temperature and high pressure. Although the injection molding technology outperforms in fabrication efficiency, the second method cannot adopt the injection molding technology lest high temperature and high pressure damage the electronic elements and the wires. Since the injection molding technology is unavailable to the second method, the second method adopts other resin-filling technologies. Before the resin-filling process, the electronic elements and the wires are placed into the mold. Thus, many gaps exist between the electronic elements and the wires and/or between the wires. Then, incomplete filling of resin may occur during the resin-filling process. Consequently, the fabrication process needs many steps to overcome the abovementioned problem, which not only makes the fabrication process troublesome and time-consuming but also makes the consistency and stability of products hard to control. Therefore, the second method is somewhat unfavorable to mass production.

Accordingly, the present invention proposes a mold for fabricating an integrated endoscopic probe, an integrated endoscopic probe, and a method for fabricating the same to overcome the abovementioned problems.

### SUMMARY OF THE INVENTION

Considering the abovementioned problems, the primary objective of the present invention is to provide an integrated endoscopic probe and a fabrication method thereof to reduce the size of the endoscopic distal tip, improve the fabrication efficiency, enhance the fabrication stability, and decrease the fabrication cost.

Another objective of the present invention is to provide a mold to minimize an endoscopic distal tip, increase the output in a single batch production, and enhance the product quality.

To achieve the abovementioned objectives, the present invention provides a mold for fabricating an integrated endoscopic probe, which is fabricated into a single-piece structure, and which comprises a body, a cover, and a protrusion member. The body has a top surface and a bottom surface, which are opposite to each other. The body also has a mold cavity, which penetrates the top surface and the bottom surface and has an inner wall surface. The cover is formed on the top surface and has a contact surface, which faces the interior of the mold cavity and is used to shield a portion of the opening of the mold cavity, whereby to partition the mold cavity into a placement region and a resin-filling region. The protrusion member has a fixed end and a free end, which are opposite to each other. A portion of the fixed end is connected with the edge of the cover. The fixed end is extended outward from the contact surface to form the free end.

In one embodiment, the cover has an inclined surface to fulfill the requirement of reducing the size of the distal tip of the final product of the integrated endoscopic probe, wherein the inclined surface is formed inside the mold cavity.

In one embodiment, the mold further includes an annular element, which sleeves the free end of the protrusion member, whereby to make the working channel of the integrated endoscopic probe has an annular groove.

In one embodiment, the contour of the edge of the mold cavity has a circular or elliptical shape.

The present invention also provides a mold for fabricating an integrated endoscopic probe, which is fabricated into a single-piece structure, and which comprises a body, at least one cover, and a plurality of protrusion members. The body has a top surface and a bottom surface, which are opposite to each other. The body has a plurality of mold cavities, which are arranged into an array on the body and respectively penetrate the top surface and the bottom surface. Each of the plurality of mold cavities has an inner wall surface. The at least one cover is formed on the surface. The at least one cover has a contact surface, which faces the interior of the mold cavity. The contact surface shields a portion of the opening of the mold cavity, whereby to partition the mold cavity into a placement region and a resin-filling region. The plurality of protrusion members is respectively disposed inside the plurality of mold cavities. Each of the plurality of protrusion members has a fixed end and a free end, which are opposite to each other. A portion of the fixed end is connected with the edge of the at least one cover. The fixed end is extended outward from the contact surface to form the free end.

According to the abovementioned objectives, the present invention further provides a method for fabricating an integrated endoscopic probe, which comprises steps:
placing the abovementioned mold on a support tray, wherein the bottom of the mold contacts the surface of the support tray.
placing a light source-sensing module inside the placement region, wherein the bottom of the light source-sensing module contacts the surface of the support tray.
filling a first resin material into the resin-filling region to encapsulate the light source-sensing module and curing the first resin material.
removing the mold and the support tray to form an endoscopic distal tip having a working channel; and
soldering an electric-connection cables to an electric-connection pad on the bottom of the endoscopic distal tip to form an integrated endoscopic probe.

In one embodiment, a plurality of molds is placed on the support tray; the number of the light source-sensing modules is the same as the number of the molds, and each mold cavity has a light source-sensing module.

In one embodiment, the mold further includes a first annular element, which sleeves the free end of the protrusion member, whereby to make the working channel of the integrated endoscopic probe has an annular groove. Alternatively, the surface of the support tray has a second annular element, which sleeves the free end of the protrusion member, whereby to make the working channel of the integrated endoscopic probe has an annular groove.

After the mold is removed from the support tray, a working channel tube is disposed in the working channel, wherein one end of the working channel tube is pressed against and connected with the annular groove.

The abovementioned soldering step further includes a step: using a second resin material to fix the electric-connection cables and the electric-connection pads and joints, whereby to enhance adherence of the electric-connection cables.

According to the abovementioned objectives, the present invention further provides an integrated endoscopic probe, which comprises an endoscopic distal tip and electric-connection cables. The endoscopic distal tip further comprises a light source-sensing module and a first resin layer. The light source-sensing module includes an image sensor and a light source module. The light source module is disposed on two sides of the image sensor module. The bottom of the light source-sensing module has at least one electric-connection pad, which is respectively connected with the image sensor and the light source module. The first resin layer encapsulates the light source-sensing module. The first resin layer has a working channel, a first surface and a second surface, wherein the first surface and the second surface are opposite to each other. The working channel neighbors the light source-sensing module and penetrates the first surface and the second surface. The image sensor protrudes from the first surface. The electric-connection pads are on the second surface. One end of the electric-connection cables are electrically connected with the electric-connection pads. A second resin material is used to fix the electric-connection cables and the electric-connection pads and solder joints to enhance adherence of the electric-connection cables.

The integrated endoscopic probe further comprises a working channel tube, which is disposed inside the working channel. A portion of the interior of the working channel has an annular groove, which is used to connect with the working channel tube.

The first resin layer and the working channel have a chamfering structure to form a chamfering structure.

Therefore, the present invention can minimize the endoscopic distal tip, whereby to reduce the size of the endoscopic probe, enhance the fabrication efficiency, lower the fabrication cost, and promote the reliability and stability of products.

The objective, technologies, features and advantages of the present invention will become apparent from the following description in conjunction with the accompanying drawings wherein certain embodiments of the present invention are set forth by way of illustration and example.

### BRIEF DESCRIPTION OF THE DRAWINGS

The foregoing conceptions and their accompanying advantages of this invention will become more readily appreciated after being better understood by referring to the following detailed description, in conjunction with the accompanying drawings, wherein:

FIG. 1A is Diagram 1 schematically showing a first embodiment of a mold of the present invention.

FIG. 1B is Diagram 2 schematically showing the first embodiment of a mold of the present invention.

FIG. 1C is Diagram 3 schematically showing the first embodiment of a mold of the present invention.

FIG. 1D is a diagram schematically showing that the molds of the first embodiment are arranged into an array.

FIG. 2A is a diagram schematically showing a second embodiment of a mold of the present invention.

FIG. 2B and FIG. 2C are sectional views of the mold of the second embodiment.

FIG. 3 is a diagram schematically showing a third embodiment of a mold of the present invention.

FIG. 4 is a diagram schematically showing a fourth embodiment of a mold of the present invention.

FIG. 5 is a flowchart of a method for fabricating an integrated endoscopic probe according to one embodiment of the present invention.

FIGS. 6-8 are Diagrams 1-3 schematically showing the fabrication process of FIG. 5, wherein the mold of FIG. 1D is used.

FIG. 9 is a diagram schematically showing the fabrication process of FIG. 5, wherein the mold of FIG. 3 is used.

FIGS. 10-12 are Diagrams 1-3 schematically showing the fabrication process of FIG. 5, wherein the mold of FIG. 4 is used.

FIG. 13 a diagram schematically showing the fabrication process of a light source-sensing module according to one embodiment of the present invention.

FIG. 14A and FIG. 14B are perspective views schematically showing a first embodiment of an integrated endoscopic probe of the present invention.

FIG. 15A and FIG. 15B are perspective views schematically showing a second embodiment of an integrated endoscopic probe of the present invention.

FIG. 16A and FIG. 16B are perspective views schematically showing a third embodiment of an integrated endoscopic probe of the present invention.

### DESCRIPTION OF THE PREFERRED EMBODIMENTS

The embodiments of the present invention will be further demonstrated in detail hereinafter in cooperation with the corresponding drawings. In the drawings and the specification, the same numerals represent the same or the like elements as much as possible. For simplicity and convenient labelling, the shapes and thicknesses of the elements may be exaggerated in the drawings. It is easily understood: the elements belonging to the conventional technologies and well known by the persons skilled in the art may be not particularly depicted in the drawings or described in the specification. Various modifications and variations made by the persons skilled in the art according to the contents of the present invention are to be included by the scope of the present invention.

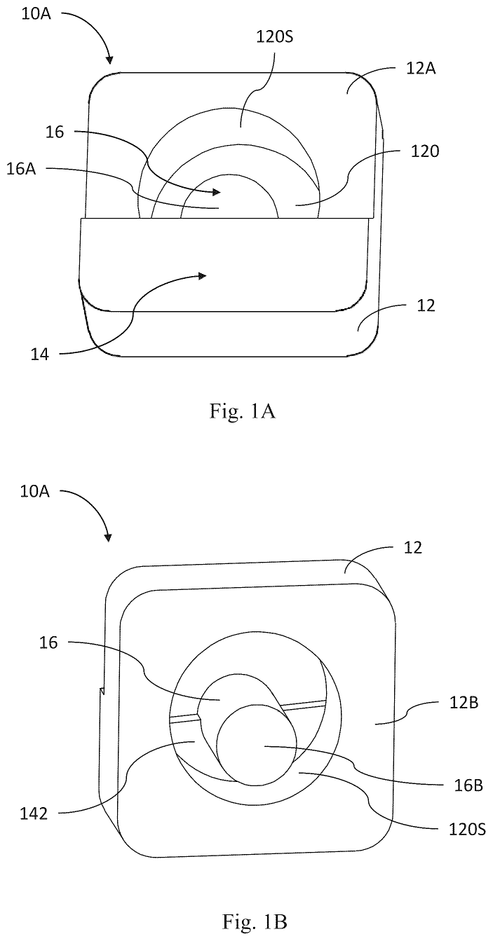

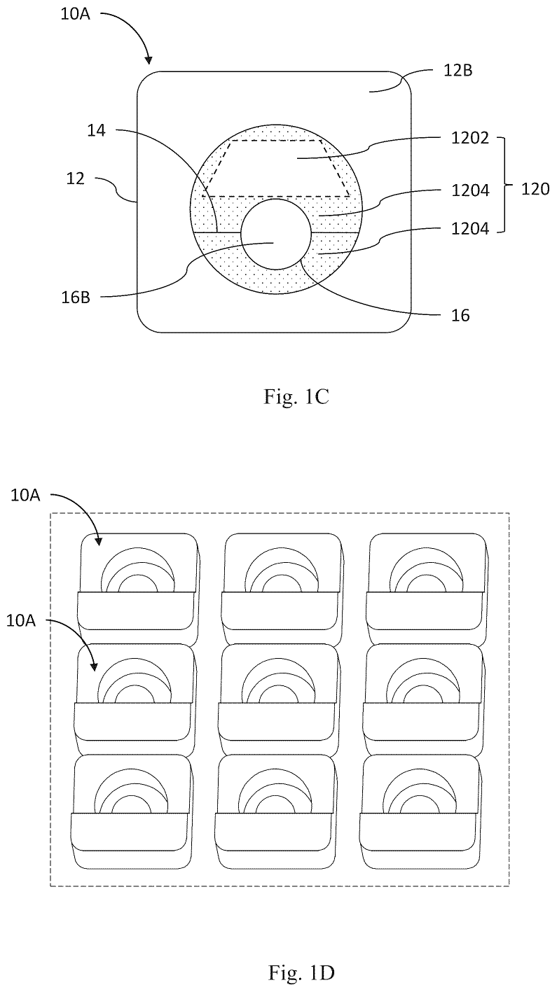

Refer to FIG. 1A-1C. The mold 10A for fabricating an integrated endoscopic probe comprises a body 12, a cover 14, and a protrusion member 16. The body 12 has a top surface 12A and a bottom surface 12B, which are opposite to each other. The body 12 has a mold cavity 120, which penetrates the top surface 12A and the bottom surface 12B. The mold cavity 120 has an inner wall surface 120S. The cover 14 is formed on the top surface 12A. A contact surface 142 of the cover 14 is the bottom of the mold cavity 120. The contact surface 142 faces the interior of the mold cavity 120, used to shield a portion of the opening of the mold cavity 120 and partition the mold cavity 120 into a placement region 1202 and a resin-filling region 1204. The protrusion member 16 is disposed inside the mold cavity 120 and formed on the cover 14. The protrusion member 16 has a fixed end 16A and a free end 16B, which are opposite to each other. The bottom of a portion of the fixed end 16A is disposed on the cover 14. The fixed end 16A is extended outward from the contact surface 142 of the cover 14 to form the free end 16B inside the mold cavity 120.

Refer to FIG. 1D simultaneously. While the plurality of molds 10A is arranged into an array for fabricating the integrated endoscopic probes, a great quantity of integrated endoscopic probes may be fabricated in a single batch. Therefore, the present invention may decrease the fabrication time and increases the yield.

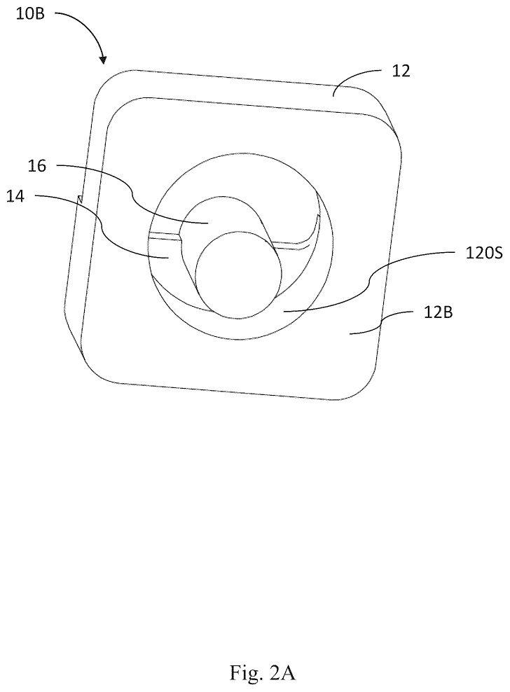

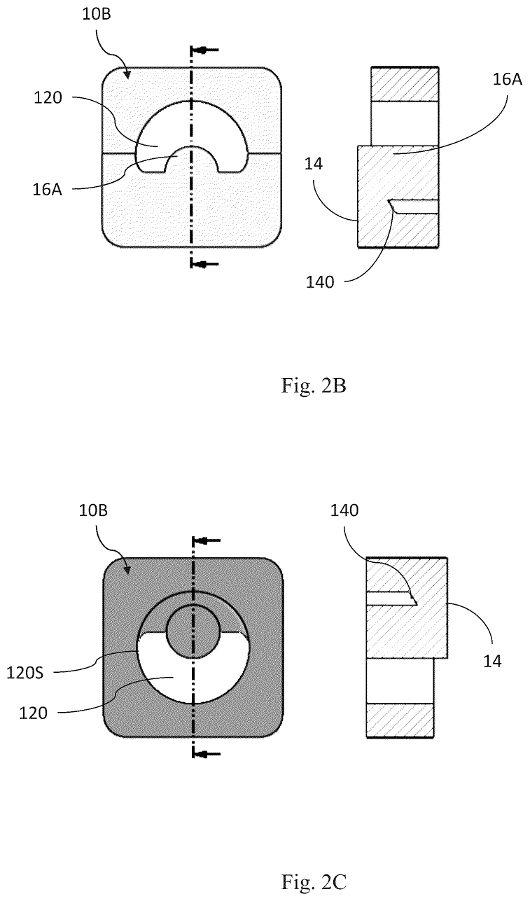

Refer to FIGS. 2A-2C. The mold 10B of the second embodiment is different from the mold 10A of the first embodiment in that the cover 14 has an inclined surface 140. The inclined surface 140 is formed inside the mold cavity 120. The inclined surface 140 is inclined from the fixed end 16A of the protrusion member 16 toward the inner wall surface 120S of the mold cavity 20. Via the mold 10B, a miniaturized endoscopic distal tip having a chamfering structure obtained.

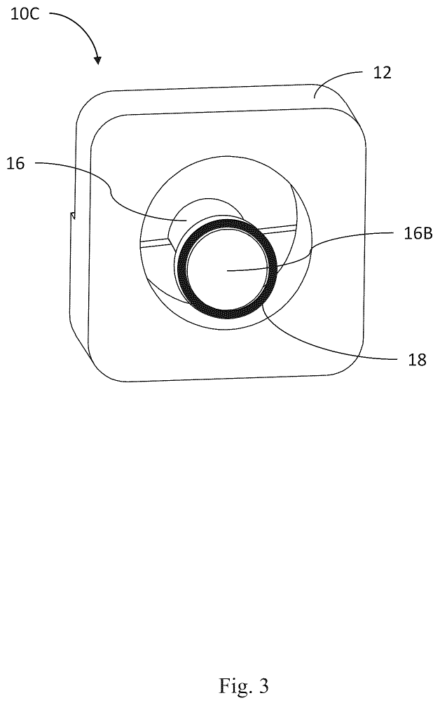

Refer to FIG. 3. The mold 10C of the third embodiment is different from the mold 10A of the first embodiment in that the mold 10C further includes a first annular element 18. The first annular element 18 sleeves the free end 16B of the protrusion member 16, used to form an annular groove in fabricating the integrated endoscopic probe. The working channel tube will be pressed against and connected to the annular groove.

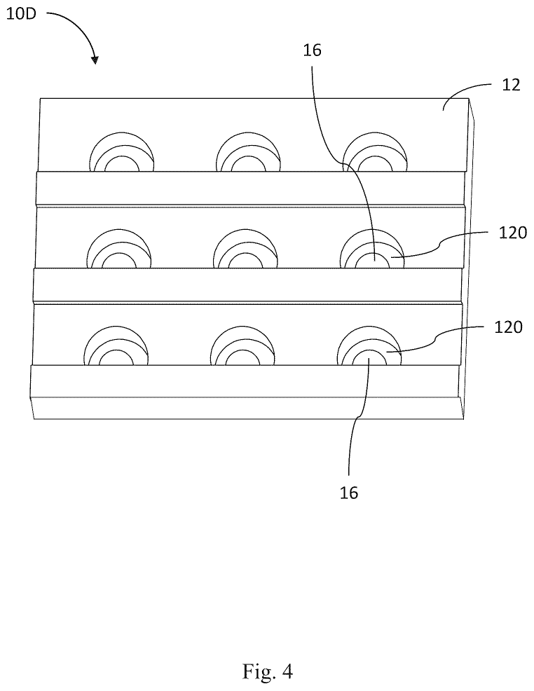

Refer to FIG. 4. The mold 10D of the fourth embodiment is different from the mold 10A of the first embodiment in that a plurality of mold cavities 120 is formed on the body 12 of the mold 10D and arranged into an array on the body 12. A plurality of protrusion members 16 is respectively disposed inside the plurality of mold cavities 120. Thereby, a plurality of integrated endoscopic distal tips may be fabricated in a single batch. Therefore, is promoted the yield and consistency of products.

The contour of the edge of the mold cavity 120 may be a circular or elliptical shape.

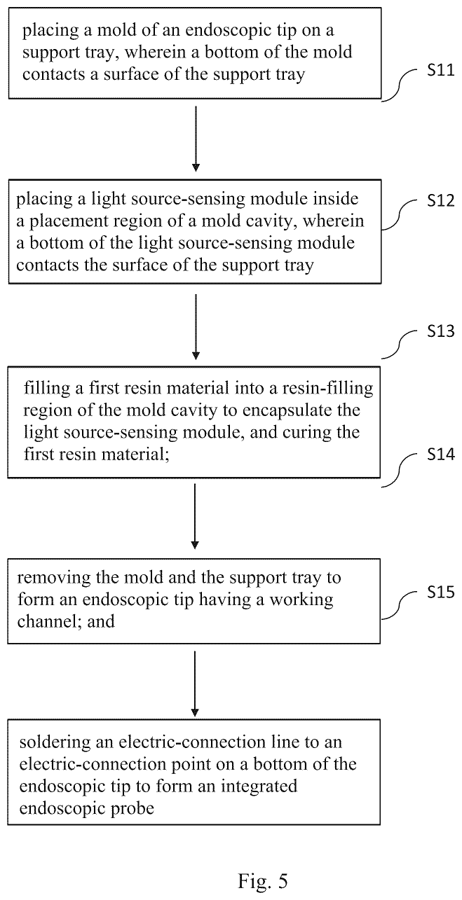

Refer to FIG. 5. The method for fabricating an integrated endoscopic probe comprises.

Step S11: placing a mold on a support tray, wherein the bottom surface of the mold contacts the surface of the support tray.

Step S12: placing a light source-sensing module in the placement region of a mold cavity of the mold, wherein the bottom of the light source-sensing module contacts the surface of the support tray.

Step S13: filling a first resin material into the resin-filling region of the mold cavity to encapsulate the light source-sensing module and curing the first resin material.

Step S14: removing the mold and the support tray to form an endoscopic distal tip having a working channel; and

Step S15: soldering an electric-connection cable to an electric-connection pad on the bottom of the endoscopic distal tip to form an integrated endoscopic probe.

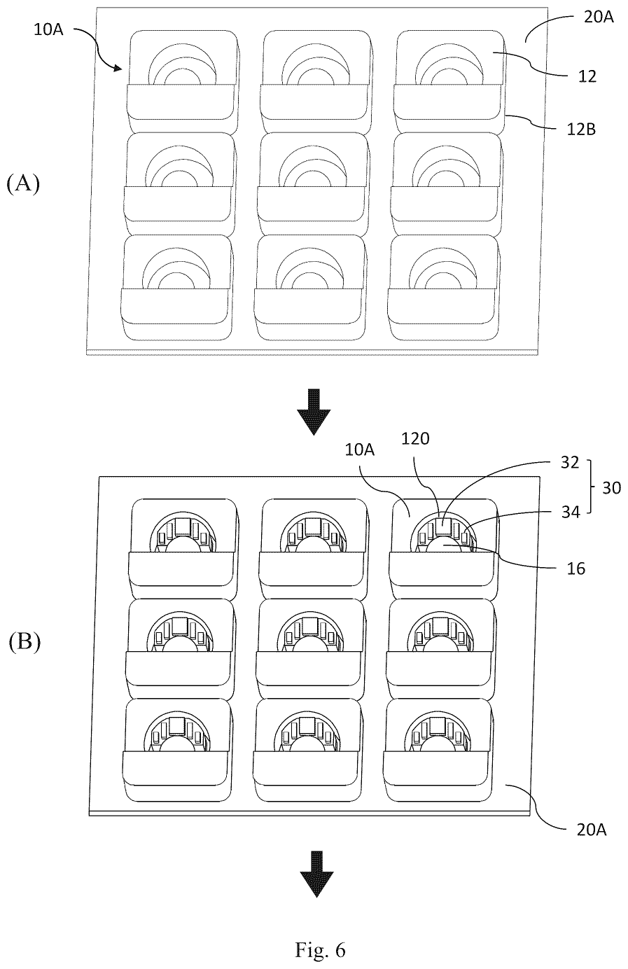

Refer to FIGS. 6-8 for further explaining the process in FIG. 5. The mold 10A in FIG. 1D is used as the exemplification below.

Refer to FIG. 6(A). In Step S11, a plurality of molds 10A is used. The plurality of molds 10A is arranged into an array on the support tray 20A. The bottom surface 12B of the body 12 of the mold 10A contacts the surface of the support tray 20A. The support tray 20A may be a thermal tape.

Refer to FIG. 6(B). In Step S12, the light source-sensing module 30 is placed in the placement region 1202 of the mold cavity 120 of each mold 10A. The bottom of the light source-sensing module 30 contacts the surface of the support tray 20A. The quantity of the light source-sensing module 30 is the same as the quantity of the mold cavities 120. The light source-sensing module 30 is disposed in the mold cavity 120 of each mold 10A. The light source-sensing module 30 includes an image sensor 32 and a light source module 34. The light source modules 34 are disposed on two sides of the image sensor 32.

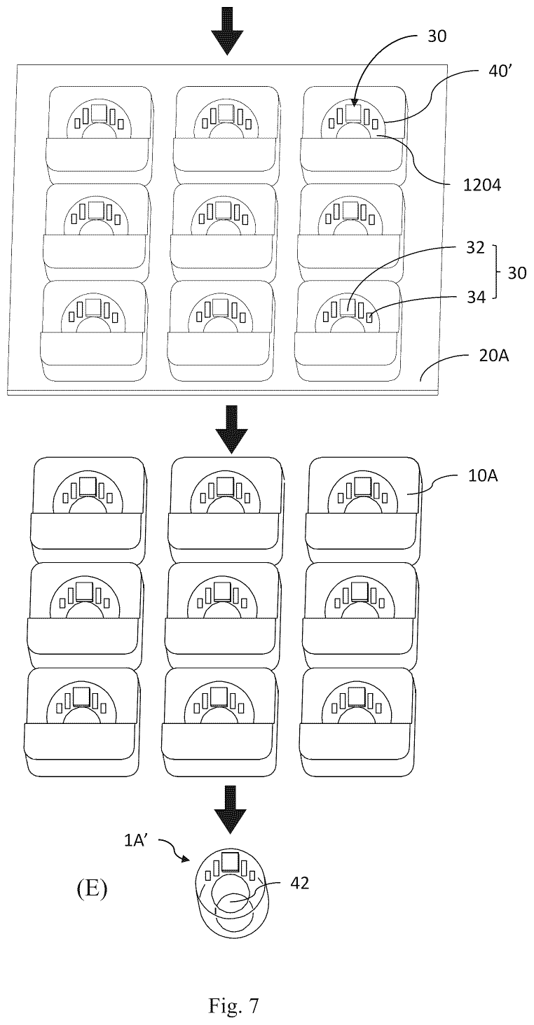

Refer to FIG. 7(C). In Step S13, a first resin material 40′ is filled into the resin-filling region 1204 to encapsulate the light source-sensing module 30, wherein an appropriate first resin material 40′ is filled into the mold cavity 120 of the mold 10A to fully encapsulate the image sensor 32 and the light source module 34. After the first resin material 40′ has encapsulated the image sensor 32 and the light source module 34, the first resin material 40′ is thermally cured or UV cured. If the structure of the image sensor 32 and the light source module 34 has regions where UV curing is unlikely to perform, a thermal-setting resin material and a thermal-setting process may be adopted to encapsulate the image sensor 32 and the light source module 34. Filling and curing resin material in a batch of products not only solves the problem of assembling a single piece of endoscopic distal tip but also greatly increases the fabrication efficiency. Further, the quality and consistency of products is maintained.

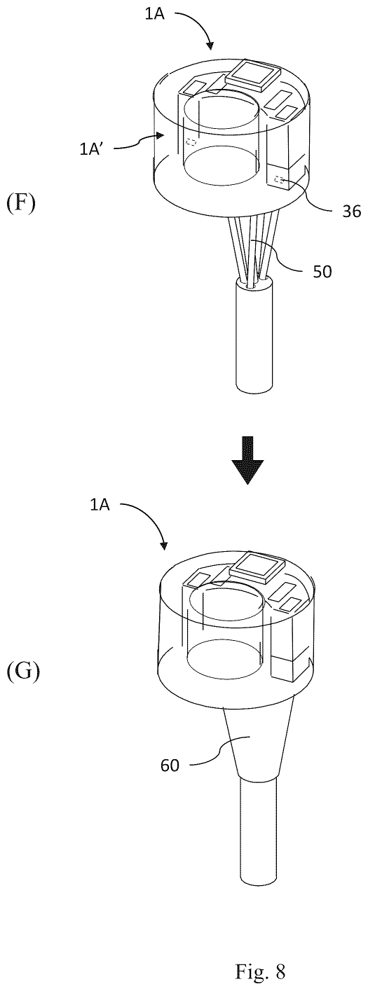

Refer to FIG. 7(D) and FIG. 7(E). In Step S14, the support tray 20A and the mold 10A are removed to form an endoscopic distal tip 1A′ having a working channel 42. Refer to FIG. 8(F). In Step S15, an electric-connection cable 50 is soldered to electric-connection pads 36 on the bottom of the endoscopic distal tip 1A′ to form an endoscopic probe 1A. During fabrication, the electric-connection pads 36 on the bottom of the endoscopic distal tip 1A′ are attached to the support tray 20A, wherein the support tray 20A is a thermal tape or any other suitable tape. Therefore, the electric-connection pads 36 are not covered by resin or other materials. Thus, the soldering of the electric-connection cable and the electric-connection pads may be performed by a soldering apparatus easily. Then, the signals may be transmitted from the image sensor to a signal processor. As the bottom of the endoscopic distal tip 1A′ is a planar structure having solder pads (the electric-connection pads 36), a soldering apparatus may be used to perform the soldering process. Thus, the efficiency of soldering is greatly increased.

Refer to FIG. 8(G). After soldering, a second resin material 60 may be used to fix the electric-connection cable 50 and the electric-connection pads 36 to enhance the adherence of the electric-connection cable 50 and prevent external force from damaging the soldering joint structure. In the present invention, the electric-connection cable 50 is disposed outside the endoscopic distal tip 1A′. Therefore, the present invention is exempted from the problem of incomplete resin filling, which is caused by the gaps of the electric-connection cable 50. Hence, the present invention can enhance the reliability of products.

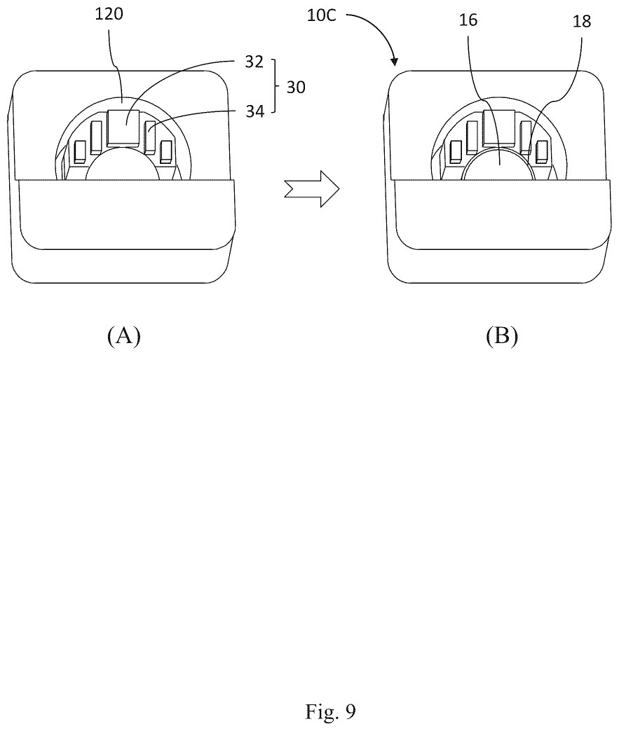

Refer to FIG. 3 and FIG. 9 for further explaining the method for fabricating an integrated endoscopic probe shown in FIG. 5. The mold 10C in FIG. 3 is used as the exemplification below. Refer to FIG. 9(A). In Step S12, the light source-sensing module 30 is placed into the mold cavity 120. Refer to FIG. 9(B). The mold 10C has a first annular element. The first annular element sleeves the free end 16B of the protrusion member 16, whereby the succeeding process may generate a working channel having an annular groove.

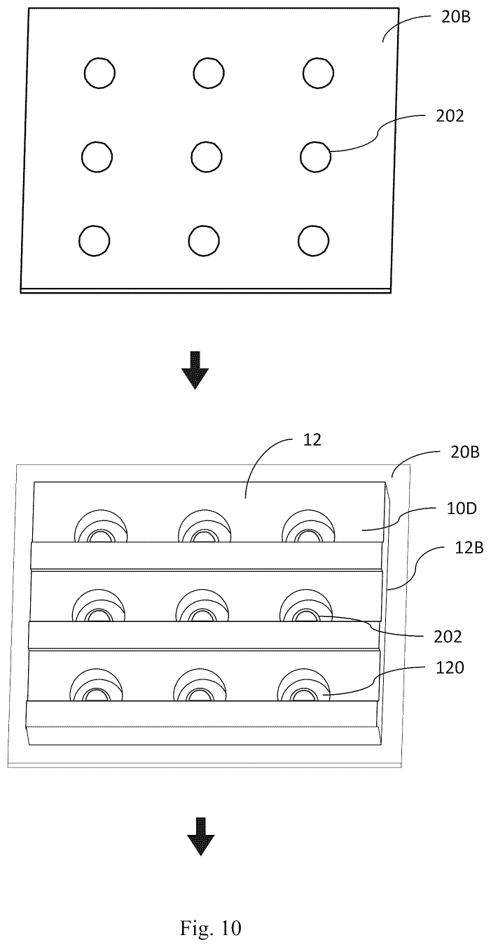

Refer to FIGS. 10-12 for further explaining the method for fabricating an integrated endoscopic probe shown in FIG. 5. Herein, diagrams are used to schematically show the fabrication process. The mold 10D in FIG. 4 is used as the exemplification below. Refer to FIG. 10(A). A second annular element 202 is disposed on an area of the support tray 20B, which is corresponding to the protrusion member 16. The quantity of the second annular elements 202 is the same as the quantity of the protrusion members 16. The second annular members 202 may sleeve the free ends 16B of the protrusion members 16.

Refer to FIG. 10(B). In Step S11, the mold 10D is placed on the support tray 20B, and the bottom surface 12B of the mold 10D contacts the surface of the support tray 20B. The mold 10D has a plurality of mold cavities 120. A plurality of protrusion members 16 is respectively disposed inside the plurality of mold cavities 120. The mold cavities 120 are arranged into an array on the body 12.

Refer to FIG. 11(C) and FIG. 11(D). In Step S12 and Step S13, a plurality of light source-sensing modules 30 is disposed inside the plurality of mold cavities 120; a first resin material 40′ is filled into the mold cavities 120 for encapsulating the light source-sensing modules 30; then, the first resin material 40′ is cured.

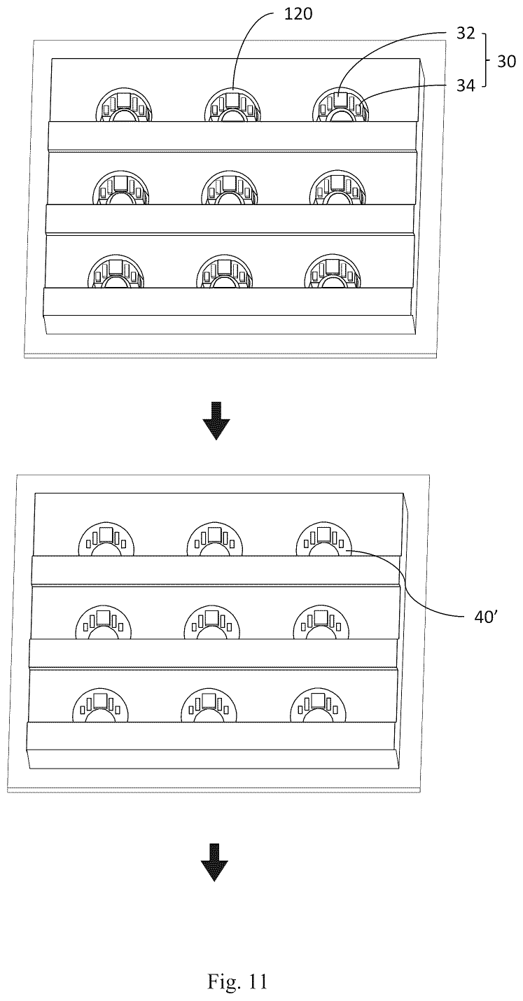

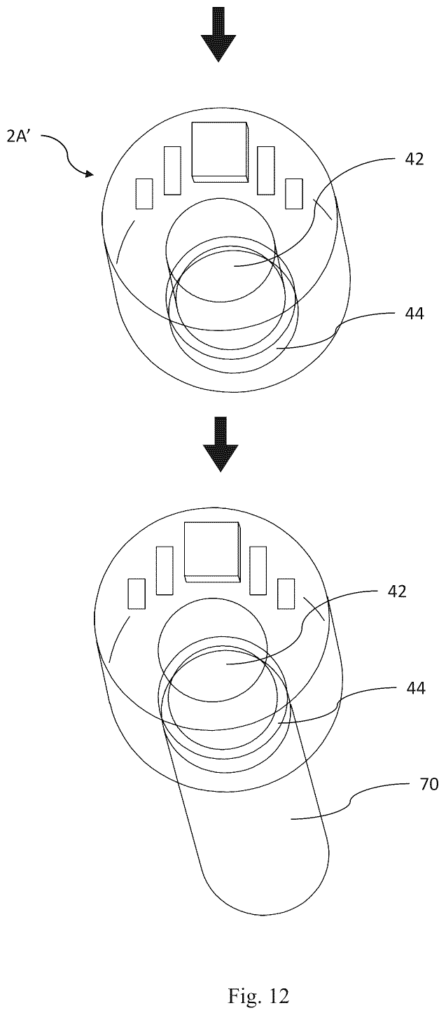

Refer to FIG. 12(E) and FIG. 12(F). In Step S14, the mold and the support tray are removed to form an endoscopic distal tip 2A′ having a working channel 42, wherein the interior of the working channel 42 has an annular groove 44, which is to be pressed against and connected with a working channel tube 70.

As shown in FIG. 12(F), the working channel tube 70 is disposed inside the working channel 42, and one end of the working channel tube 70 is pressed against and connected with the annular groove 44. Thereby, the working channel 42 may be completely connected with the endoscopic distal tip 2A′ to provide more endoscopic functions.

It should be mentioned: the first annular element and the second annular element 202 are exclusive to each other. In other words, the method of fabrication an integrated endoscopic probe can only adopt either the first annular element or the second annular element 202.

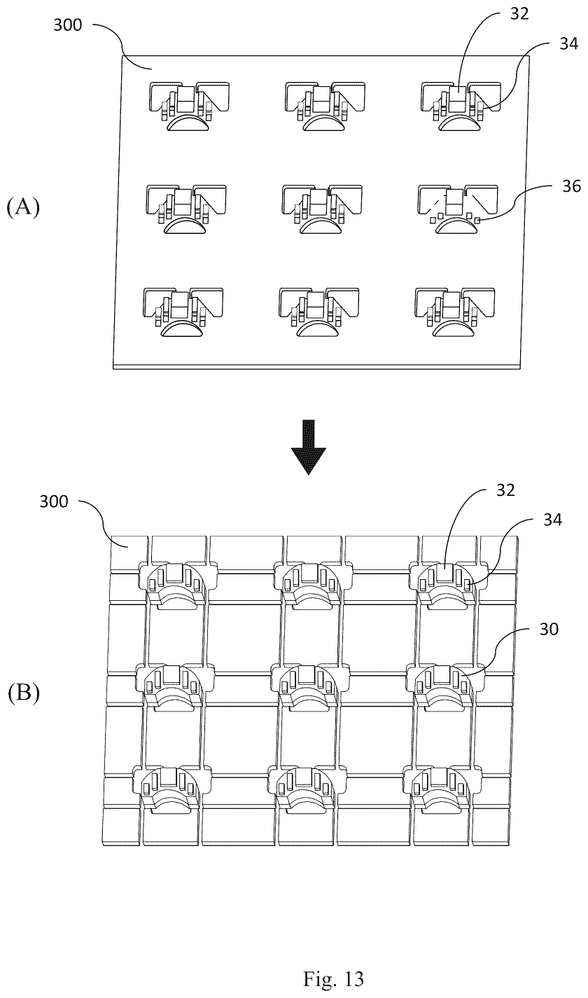

Refer to FIG. 13 for explaining the Step S12 of the method for fabricating an integrated endoscopic probe shown in FIG. 5. The light source-sensing module 30 may be encapsulated beforehand. As shown in FIG. 13(A), the image sensors 32 and the light source modules 34 are placed on the electric-connection pads 36 of a substrate 300 in advance. As shown in FIG. 13(B), the image sensors 32 and the light source modules 34 are encapsulated; then the substrate 300 is cut to form the light source-sensing modules 30. Next, the light source-sensing modules 30 are placed inside the mold.

The light source module 34 may be a light-emitting diode (LED) or an optical fiber light source. The LED light source may be a single-wavelength light source or a mixed-wavelength light source, such as a white light source, a monochromatic RGB light source, or an infrared light source.

In the description of the mold for fabricating an integrated endoscopic probe and the method for the same, the structure and concept of the integrated endoscopic probe is also described simultaneously. However, the structure of the integrated endoscopic probe will be further described with perspective views thereof to demonstrate the structure in detail below.

Refer to FIG. 14(A) and FIG. 14(B) for a first embodiment of the integrated endoscopic probe of the present invention. The integrated endoscopic probe 1A comprises an endoscopic distal tip 1A′ and an electric-connection cable. The endoscopic distal tip 1A′ includes a light source-sensing module 30 and a first resin layer 40. The light source-sensing module 30 includes an image sensor 32 and a light source module 34. The light source modules 34 are disposed on two sides of the image sensor 32. The bottom of the light source-sensing module 30 has electric-connection pads 36. The electric-connection pads 36 are respectively connected with the image sensor 32 and the light source module 34. The first resin layer 40 encapsulates the light source-sensing module 30. The first resin layer 40 has a working channel 42, a first surface 40A, and a second surface 40B, wherein the first surface 40A and the second surface 40B are opposite to each other. The working channel 42 neighbors the light source-sensing module 30 and penetrates through the first surface 40A and the second surface 40B. The image sensor 32 protrudes from the first surface 40A. The electric-connection pads 36 are arranged on the second surface 40B. One end of the electric-connection cable 50 is electrically connected the electric-connection pads 36. A second resin material 60 fixes the electric-connection cable 50 and the electric-connection pads36 to enhance the adherence of the electric-connection cable 50.

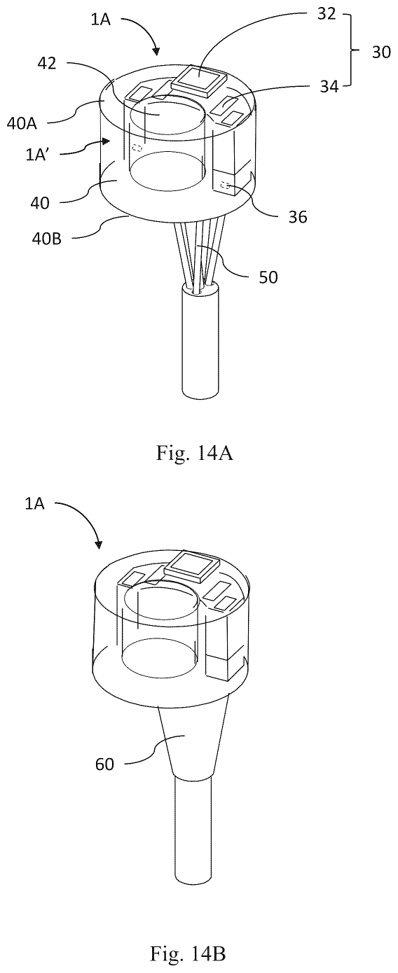

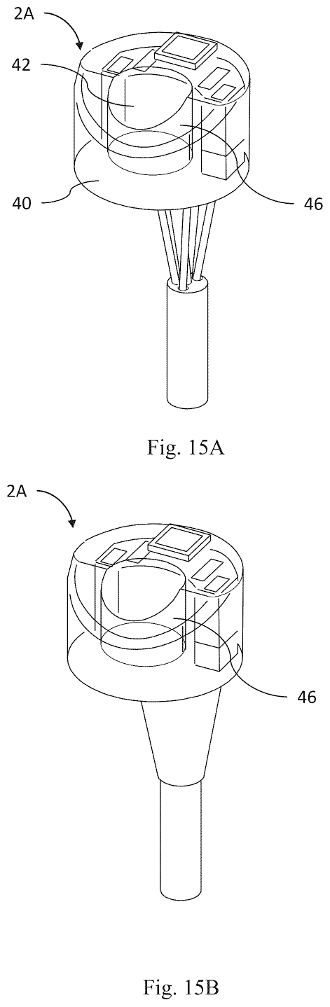

Refer to FIG. 15A and FIG. 15B for a second embodiment of the integrated endoscopic probe of the present invention. The integrated endoscopic probe 2A of the second embodiment is different from the integrated endoscopic probe 1A of the first embodiment in that the first resin layer 40 and the working channel 42 has a chamfering structure 46 in the second embodiment. The chamfering structure 46 forms a chamfering structure, which favors further reducing the size of the endoscopic distal tip.

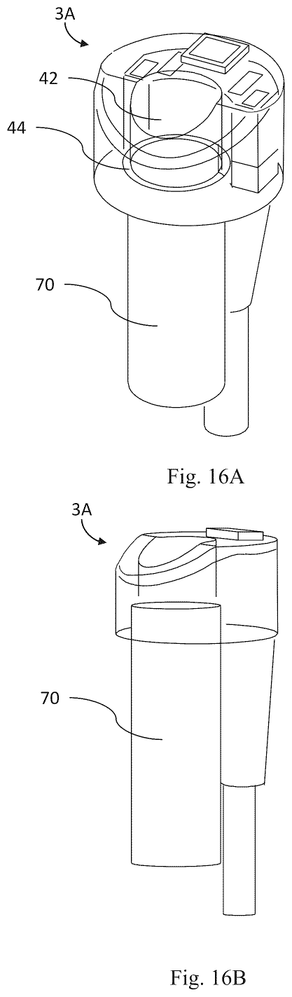

Refer to FIG. 16A and FIG. 16B for a third embodiment of the integrated endoscopic probe of the present invention. The integrated endoscopic probe 3A of the third embodiment is different from the integrated endoscopic probe 2A of the second embodiment in that the integrated endoscopic probe 3A further includes a working channel tube 70. The working channel tube 70 is disposed inside the working channel 42. A portion of the interior of the working channel 42 has an annular groove 44, which is used in the connection with the working channel tube 70. The working channel 42 is in form of a cylindrical shape or an elliptical column shape.

In conclusion, the present invention can increase the fabrication efficiency greatly, maintain the consistency and stability of products, favor mass production, and promote the reliability of the fabrication process. Further, the present invention can fabricate an integrated endoscopic probe having a further miniaturized size to provide the users more convenient application.

The embodiments described above are to exemplify and demonstrate the present invention but not to limit the scope of the present invention. Any equivalent substitution or variation according to the specification or claims of the present invention is to be also included by the scope of the present invention.

While the invention is susceptible to various modifications and alternative forms, a specific example thereof has been shown in the drawings and is herein described in detail. It should be understood, however, that the invention is not to be limited to the form disclosed, but to the contrary, the invention is to cover all modifications, equivalents, and alternatives falling within the appended claims.

1. A mold for fabricating an integrated endoscopic probe, comprising
a body, having a top surface and a bottom surface, which are opposite to each other, and a mold cavity, which penetrates the top surface and the bottom and has an inner wall surface;
a cover, formed on the top surface and has a contact surface functioning as a bottom of the mold cavity, wherein the contact surface faces interior of the mold cavity, shields a portion of an opening of the mold cavity, and partitions the mold cavity into a placement region and a resin-filling region; and
a protrusion member, disposed inside the mold cavity, formed on the cover, and having a fixed end and a free end, which are opposite to each other, wherein a portion of the fixed end is located at the cover; the fixed end is extended outward from the contact surface of the cover to form the free end inside the mold cavity.

2. The mold for fabricating an integrated endoscopic probe according to claim 1, wherein the cover has an inclined surface; the inclined surface is formed inside the mold cavity.

3. The mold for fabricating an integrated endoscopic probe according to claim 1, further comprising a first annular element, wherein the first annular element sleeves the free end of the protrusion member.

4. The mold for fabricating an integrated endoscopic probe according to claim 1, wherein a contour of an edge of the mold cavity has a circular shape or an elliptical shape.

5. A mold for fabricating an integrated endoscopic probe, comprising
a body, having a top surface and a bottom surface, which are opposite to each other, and a plurality of mold cavities, which are arranged into an array on the body and respectively penetrate the top surface and the bottom surface, wherein each of the plurality of mold cavities has an inner wall surface;
a plurality of covers, formed on the top surface, wherein each of the covers has a contact surface functioning as a bottom of the mold cavity; the contact surfaces respectively face interior of the plurality of mold cavities and respectively partially shield openings of the plurality of mold cavities; each contact surface partitions the mold cavity into a placement region and a resin-filling region, which are adjacent to each other; and
a plurality of protrusion members, respectively disposed inside the plurality of mold cavities, wherein each of the plurality of protrusion members is formed one of the plurality of covers and has a fixed end and a free end, which are opposite to each other; a bottom of the fixed end is partially located at the cover; the fixed end is extended outward from the contact surface of the cover to form the free end.

6. A method for fabricating an integrated endoscopic probe, comprising steps:
placing the mold of claim 1 on a support tray, wherein a bottom of the mold contacts a surface of the support tray;
placing a light source-sensing module inside the placement region, wherein a bottom of the light source-sensing module contacts the surface of the support tray;
filling a first resin material into the resin-filling region to encapsulate the light source-sensing module, and curing the first resin material;
removing the mold and the support tray to form an endoscopic distal tip having a working channel; and
soldering an electric-connection cable to an electric-connection pad on a bottom of the endoscopic distal tip to form an integrated endoscopic probe.

7. The method for fabricating an integrated endoscopic probe according to claim 6, wherein a plurality of molds is disposed on the support tray; a count of the light source-sensing modules is the same as a count of the molds; the light source-sensing module is disposed inside the mold cavity of each of the molds.

8. The method for fabricating an integrated endoscopic probe according to claim 6, wherein the cover is recessed toward the mold cavity to form an inclined surface; the cover has a first lateral surface and a second lateral surface, which are adjacent to each other; the first lateral surface is connected with the inner wall surface of the mold cavity; the second lateral surface is connected with the fixed end of the protrusion member.

9. The method for fabricating an integrated endoscopic probe according to claim 6, wherein the mold includes a first annular element; the first annular element sleeves the free end of the protrusion member to make the working channel have an annular groove.

10. The method for fabricating an integrated endoscopic probe according to claim 6, wherein a second annular element is disposed on the surface of the support tray; the second annular element sleeves the free end of the protrusion member to make the working channel have an annular groove.

11. The method for fabricating an integrated endoscopic probe according to claim 9, wherein after the mold and the support tray are removed, a working channel tube is disposed in the working channel; one end of the working channel tube is pressed against and connected with the annular groove.

12. The method for fabricating an integrated endoscopic probe according to claim 6, wherein the step of curing the first resin material includes thermally curing the first resin material or UV curing the first resin material; the first resin material includes a thermosetting resin material.

13. The method for fabricating an integrated endoscopic probe according to claim 6, wherein the soldering step further includes a step: using a second resin material to fix the electric-connection cable and electric-connection pad after soldering to enhance adherence of the electric-connection cable.

14. The method for fabricating an integrated endoscopic probe according to claim 6, wherein the step of placing a light source-sensing module further includes steps: placing an image sensor and a light source module on a substrate and soldering the image sensor and the light source module to the electric-connection pads of the substrate; encapsulating the image sensor and the light source module; and cutting the substrate to form the light source-sensing module.

15. A method for fabricating an integrated endoscopic probe, comprising steps:
placing the mold of claim 5 on a support tray, wherein a bottom of the mold contacts a surface of the support tray;
respectively placing a plurality of light source-sensing modules on the placement regions of the plurality of mold cavities, wherein bottoms of the plurality of light source-sensing modules contact the bottom of the support tray;
filling a first resin material into the resin-filling regions of the plurality of mold cavities to respectively encapsulate the plurality of light source-sensing modules, and then curing the first resin material;
removing the mold and the support tray to respectively form endoscopic distal tips each having a working channel; and
soldering an electric-connection cable to an electric-connection pad on a bottom of the endoscopic distal tip to form an integrated endoscopic probe.

16. An integrated endoscopic probe, comprising
an endoscopic distal tip, including:
a light source-sensing module, including an image sensor and a light source module, wherein the light source modules are disposed on two sides of the image sensor module; a bottom of the light source module has an electric-connection pad, which is respectively connected with the image sensor and the light source module;
a first resin layer, encapsulating the light source-sensing module, wherein the first resin layer has a working channel, a first surface, and a second surface opposite to the first surface; the working channel neighbors the light source-sensing module and penetrates the first surface and the second surface; the image sensor protrudes from the first surface; the electric-connection pad is on the second surface; and
an electric-connection cable, wherein one end of the electric-connection cable is electrically connected with the electric-connection pads; a second resin material is used to fix the electric-connection cable and the electric-connection pads to enhance adherence of the electric-connection cables.

17. The integrated endoscopic probe according to claim 16, further comprising a working channel tube disposed inside the working channel.

18. The integrated endoscopic probe according to claim 17, wherein a portion of the working channel has an annular groove used to connect with the working channel tube.

19. The integrated endoscopic probe according to claim 16, wherein a chamfering structure is formed on the first resin layer and the working channel to function as a chamfering structure.

20. The integrated endoscopic probe according to claim 16, wherein the working channel has a cylindrical shape or an elliptical column shape.

21. The method for fabricating an integrated endoscopic probe according to claim 10, wherein after the mold and the support tray are removed, a working channel tube is disposed in the working channel; one end of the working channel tube is pressed against and connected with the annular groove.
